(19) United States
(12) Patent Application Publication
(10) Pub. No.: US 2023/0271012 A1
(43) Pub. Date: Aug. 31, 2023

(54) TREATMENT OF GAIT IMPAIRMENT USING DEEP BRAIN STIMULATION

(71) Applicant: Oxford University Innovation Limited, Oxford (GB)

(72) Inventors: Petra FISCHER, Oxford (GB); Shenghong HE, Oxford (GB); Huiling TAN, Oxford (GB); Peter BROWN, Oxford (GB)

(21) Appl. No.: 18/008,274

(22) PCT Filed: Jun. 9, 2021
(86) PCT No.: PCT/GB2021/051427

§ 371 Date: Dec. 5, 2022

(30) Foreign Application Priority Data
Jun. 11, 2020 (GB) 2008841.5

(51) Int. Cl. A61N 1/36; A61N 1/05

(52) U.S. Cl. CPC A61N 1/36067; A61N 1/0531; A61N 1/36192; A61N 1/36196; A61N 1/36175

(57) ABSTRACT

There is provided a stimulation device for treatment of gait impairment of a patient. The stimulation device is configured to apply respective stimulation signals to electrodes bilaterally implanted in two subcortical regions of the left and right hemispheres of the brain of the patient, the subcortical regions being associated with motor control. The stimulation device is configured to apply respective stimulation signals having a rate of electrical energy delivered that is modulated with alternating waveforms at a gait frequency and out of phase with each other.

The present invention relates to stimulation devices for providing a stimulation signal to a target area of a human or animal subject. In particular it relates to stimulation of subcortical regions of the brain for treatment of gait impairment.

Patients with Parkinson's can experience debilitating treatment-resistant gait disturbances. Some of the most challenging symptoms for patients with Parkinson's disease are gait and balance problems as they can cause falls, loss of mobility and strongly reduce patients' quality of life, posing a major burden to patients and their families. About half of all patients suffer from sudden motor blocks during walking, so-called freezing episodes, which are often unresponsive to medication. Rhythmic auditory or tactile cues can reduce freezing severity and improve gait rhythmicity. However, these cues have limitations as not all patients benefit from it and benefits may deteriorate over time.

Deep brain stimulation (DBS) of the subthalamic nucleus (STN) is an effective treatment for tremor, rigidity and bradykinesia in Parkinson's disease. However, it is less successful for treating gait problems. DBS often fails to relieve gait problems and can even aggravate symptoms such as freezing of gait (FOG) or imbalance.

Conventional high-frequency DBS is provided continuously and is thought to attenuate beta activity. Several reports describe changes in STN beta activity or its phase locking between hemispheres during gait. STN activity is modulated in a rhythmic pattern contralateral to stepping movements when patients perform stepping movements. Beta (20-30 Hz) activity briefly increased just after the contralateral heel strike during the stance period, resulting in alternating peaks of right and left STN activity. Auditory cueing further enhanced this alternating pattern. However, previous neurophysiological studies have fallen short of proving that the STN is causally involved in shaping the dynamics of stepping and helped organise the stepping behaviour, or was secondary and afferent to it.

It is an object of the invention to enable improved DBS for the treatment of gait impairment, for example for patients suffering from Parkinson's disease.

According to an aspect of the invention, there is provided a stimulation device for treatment of gait impairment of a patient, the stimulation device being configured to apply respective stimulation signals to electrodes bilaterally implanted in two subcortical regions of the left and right hemispheres of the brain of the patient, the subcortical regions being associated with motor control, wherein the stimulation device is configured to apply respective stimulation signals having a rate of electrical energy delivered that is modulated with alternating waveforms at a gait frequency and out of phase with each other.

It is hypothesised that the modulation of activity in subcortical regions associated with motor control (such as the STN) is indicative of such subcortical regions being directly involved in stepping control. In this case, rhythmic stimulation of subcortical regions associated with motor control with the rate of electrical energy delivered rhythmically modulated should entrain the stepping rhythm. This is achieved by alternating stimulation delivered to the two subcortical regions associated with motor control (such as the STN) between the left and right hemispheres. This has clinical significance, as experimental results indicate that the change to alternating deep brain stimulation (altDBS) could provide a novel approach to reinforce the normal stepping cycle, and may thus be superior in improving gait problems than continuous deep brain stimulation (contDBS).

In an embodiment, rhythmical modulation of the rate of electrical energy delivered can be achieved by modulating the stimulation frequency, the amplitude, the pulse width of the electrical pulses delivered, or a combination of these parameters.

In an embodiment, the stimulation signals have a stimulation frequency of at least 20 Hz, preferably at least 50 Hz. In an embodiment, the stimulation signals have a stimulation frequency of at most 180 Hz. These ranges of frequencies are preferred as being most effective for producing the desired effect in the specifically targeted subcortical regions of the brain.

In an embodiment, the stimulation signals have a stimulation frequency that is at least 20% of the frequency of a clinical stimulation signal that is clinically defined for use during continuous chronic stimulation of the targeted area. In an embodiment, the stimulation signals have a maximal stimulation frequency that is at most 140% of the frequency of a clinical stimulation signal that is clinically defined for use during continuous chronic stimulation. When prescribing DBS using known contDBS treatment methods, a physician will typically choose a preferred clinical stimulation frequency with a particular frequency. This is based on a number of factors, for example the patient's age and symptoms. It is not necessary to use exactly the clinically defined frequency to obtain the desired effect, and these preferred upper and lower limits represents frequencies that are still capable of producing the desired relief of symptoms.

In an embodiment, the alternating waveforms have a maximum rate of electrical energy delivered and a minimum rate of electrical energy delivered, the minimum rate of electrical energy delivered being 65% or less of the maximum rate of electrical energy delivered. In order to produce the advantageous effects of the present invention, it is preferred that the modulation of the stimulation signals provides a clear variation in the rate of electrical energy delivered.

In an embodiment, the alternating waveforms have a maximum rate of electrical energy delivered that is at least 100% of the rate of electrical energy delivered of a clinical stimulation signal that is clinically defined for use during continuous chronic stimulation. In an embodiment, the alternating waveforms have a maximum rate of electrical energy delivered that is at most 200% of the rate of electrical energy delivered of a clinical stimulation signal that is clinically defined for use during continuous chronic stimulation. These preferred upper and lower limits ensure that the rate of electrical energy delivered at the peak of the modulation is at least that known to be effective for contDBS, and/or avoid too high a rate of electrical energy delivery that may cause undesirable side effects for the patient.

In an embodiment, the alternating waveforms have a minimum rate of electrical energy delivered that is at most 65% of the rate of electrical energy delivered of a clinical stimulation signal that is clinically defined for use during continuous chronic stimulation. This endures there is a clear distinction between the minimum and maximum rate of energy delivered in different parts of the modulation waveform.

In an embodiment, the rate of electrical energy delivered of the alternating waveforms remains in the upper quartile of the range between the minimum rate of electrical energy delivered and maximum rate of electrical energy delivered for at least 10% of the period of the alternating waveforms, preferably for at least 20% of the period of the alternating waveforms. In an embodiment, the rate of electrical energy delivered of the alternating waveforms remains in the upper quartile of the range between the minimum rate of electrical energy delivered and maximum rate of electrical energy delivered for at most 90% of the period of the alternating waveforms, preferably for at most 70% of the period of the alternating waveforms. Any form of alternating waveform may be used to provide the modulation of the stimulation signals. These preferred lower and upper limits ensure that the waveform used delivers sufficient electrical energy at a reasonable rate to produce the desired symptom relief.

In an embodiment, the rate of electrical energy delivered of the alternating waveforms remains in the lower quartile of the range between the minimum rate of electrical energy delivered and maximum rate of electrical energy delivered for at most 90% of the period of the alternating waveforms, preferably for at most 80% of the period of the alternating waveforms. In an embodiment, the rate of electrical energy delivered of the alternating waveforms remains in the lower quartile of the range between the minimum rate of electrical energy delivered and maximum rate of electrical energy delivered for at least 10% of the period of the alternating waveforms, preferably for at least 30% of the period of the alternating waveforms. These preferred lower and upper limits ensure that the waveform used provides a clear distinction between higher and lower rate of electrical energy delivered during the cycle of the alternating waveforms, while still delivering electrical energy at a rate sufficient to produce the desired symptom relief.

In an embodiment, the stimulation device is configured to apply respective stimulation signals having the rate of electrical energy delivered modulated by modulation of an amplitude of the stimulation signals. Modulation of amplitude can be achieved using relatively simple apparatus such as switches, and so may be preferred for ease of implementation.

In an embodiment, the stimulation device is configured to apply respective stimulation signals having the rate of electrical energy delivered modulated by modulation of a stimulation frequency of the stimulation signals. In an embodiment, the stimulation device is configured to apply respective stimulation signals having the rate of electrical energy delivered that is modulated by modulation of a pulse width of the stimulation signals. Modulation of the frequency or pulse width of the stimulation signals will also vary the rate of electrical energy delivered, and may be more convenient in some embodiments depending on the implementation of the stimulation device.

In an embodiment, the stimulation device is configured to apply respective stimulation signals having the rate of electrical energy delivered modulated by modulation of at least two of: an amplitude of the stimulation signals; a stimulation frequency of the stimulation signals; and a pulse width of the stimulation signals. Modulating multiple properties of the stimulation signal simultaneously can allow greater flexibility in the control of the stimulation signal, which may be desirable for implementing more complex waveforms.

In an embodiment, the alternating waveforms are out of phase by 50% of the period of the alternating waveforms for the left and right hemispheres. In general, a person's gait involves taking steps that are out of phase by half a cycle, and so this phase difference between the modulations of the stimulation signals applied to each hemisphere to this difference can improve the effectiveness of treatment of gait impairment. However, this is not necessary, and a range of phase differences may be used in some situations depending on specific patients' needs or the design of the stimulation device.

In an embodiment, the alternating waveforms are square waveforms. Square waveforms are particularly straightforward to implement, for example by switching the stimulation signal on and off periodically. However, many other types of waveforms are possible, such as triangular waves, sawtooth waves, or sinusoidal waves, and may be preferred in certain circumstances.

In an embodiment, the stimulation signals have identical alternating waveforms. This is preferred because applying the same stimulation signal to each hemisphere of the brain produces consistent treatment over the entire gait cycle of a patient. It may also be more straightforward to implement by not requiring different waveforms to be generated for each hemisphere. However, this is not essential, in some situations it may be desirable to use different waveforms and different parameters for different electrodes, for example to compensate for differences in structure between the hemispheres of an individual's brain.

In an embodiment, the gait frequency is a desired gait frequency. In an embodiment, the desired gait frequency is a gait frequency measured in the absence of stimulation. Matching the stimulation to a desired or natural gait of the patient will provide the most effective and consistent reduction in symptoms over multiple steps.

In an embodiment, the desired gait frequency is in the physiological range for an age group of a patient. This may provide a more simplistic way to set the desired frequency, for example if measuring the natural gait of the patient is difficult, or as an initial setting prior to further observation of the patient.

In another embodiment, the stimulation device further comprises a tracking system arranged to track the gait of the patient and the stimulation device is arranged to apply stimulation signals having a rate of electrical energy delivered that is modulated with alternating waveforms at a gait frequency synchronously with the tracked gait. Such tracking allows the stimulation pattern to be aligned to the stepping rhythm as the patient starts walking.

In an embodiment, the subcortical regions are the subthalamic nuclei. As discussed above, previous studies suggest his region is associated with gait control, and so stimulating this region will produce more effective treatment.

In an embodiment, the subcortical regions are the pedunculopontine nuclei. Stimulation of these regions is also effective in treatment of gait impairment.

In an embodiment, the patient is a patient with Parkinson's Disease, Progressive Supranuclear Palsy or Multiple System Atrophy. These conditions are known to result in gait impairment, and DBS is an effective treatment for the symptoms of these conditions.

In an embodiment, the stimulation device further comprises electrodes for bilateral implantation in the two subcortical regions of the left and right hemispheres of the brain of the patient. Providing the electrodes as part of the system means they can be more specifically designed to effectively provide the stimulation signals of the invention.

According to another aspect of the invention, there is provided a method of treatment of gait impairment of a patient, the method comprising applying respective stimulation signals to electrodes bilaterally implanted in two subcortical regions of the left and right hemispheres of the brain of the patient, being regions associated with motor control, the respective stimulation signals that are amplitude modulated by alternating waveforms at a gait frequency and out of phase with each other. As discussed for the system above, this type of modulated stimulation signal provides improved reduction in gait impairment compared to contDBS.

To allow better understanding, embodiments of the present invention will now be described by way of non-limitative example with reference to the accompanying drawings, in which:

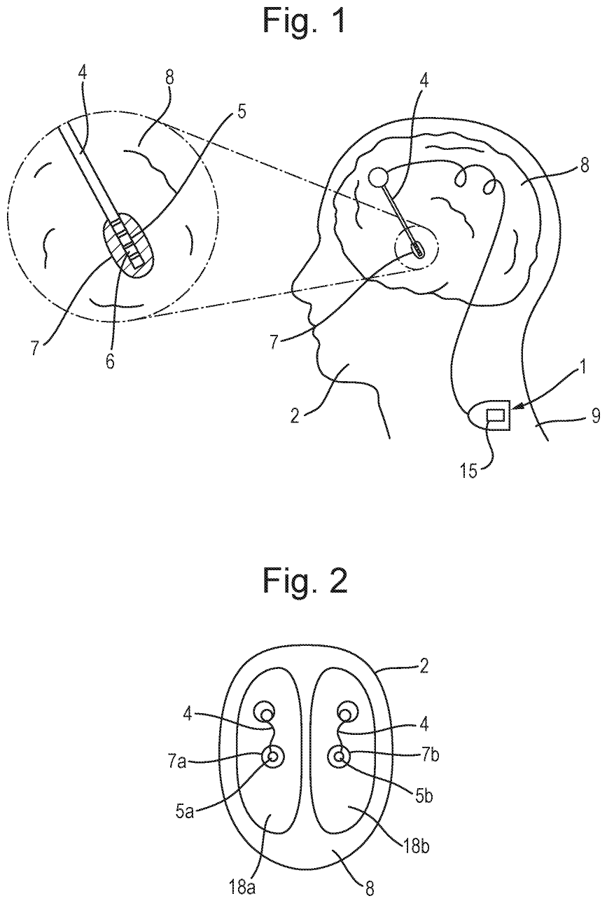

FIG. 1 is a pair of perspective views of a DBS device implanted in a patient;

FIG. 2 is a top-down perspective view illustrating implantation of electrodes in both hemispheres of the brain;

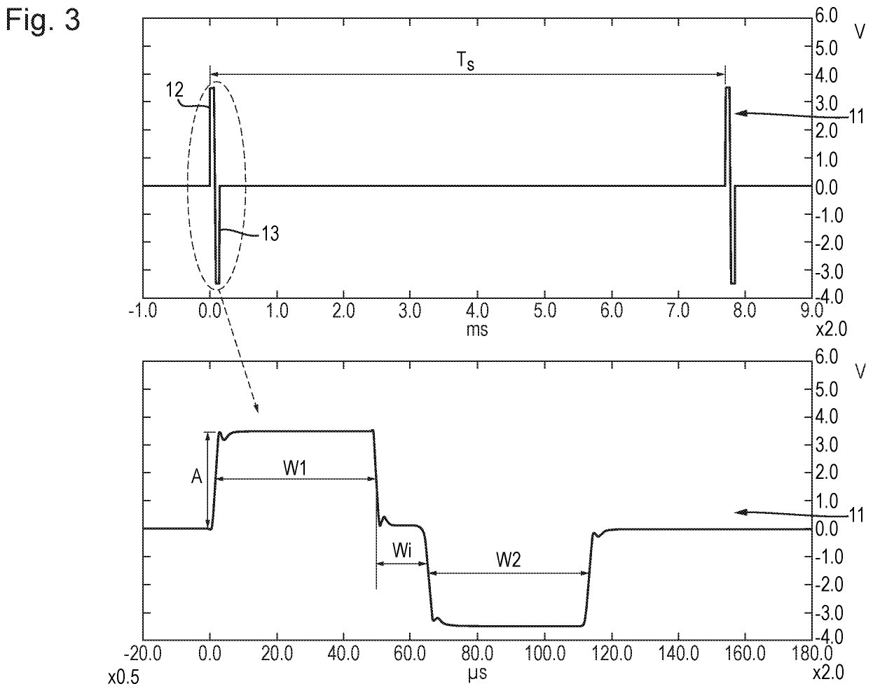

FIG. 3 is a pair of graphs illustrating a typical stimulation signal;

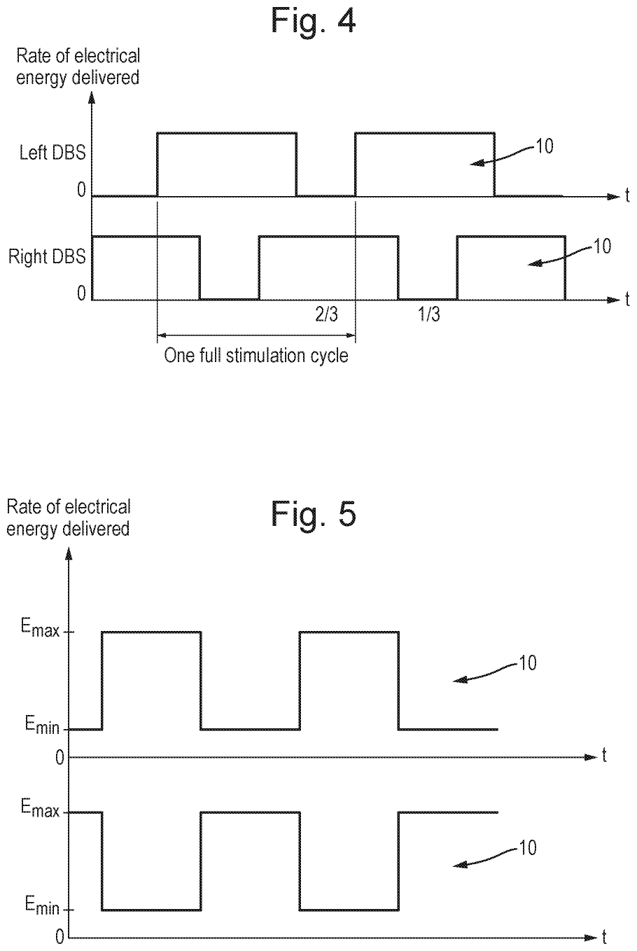

FIG. 4 shows the alternating waveform used to obtain experimental results discussed herein;

FIG. 5 illustrates an alternative alternating waveform for modulation of the rate of electrical energy delivered;

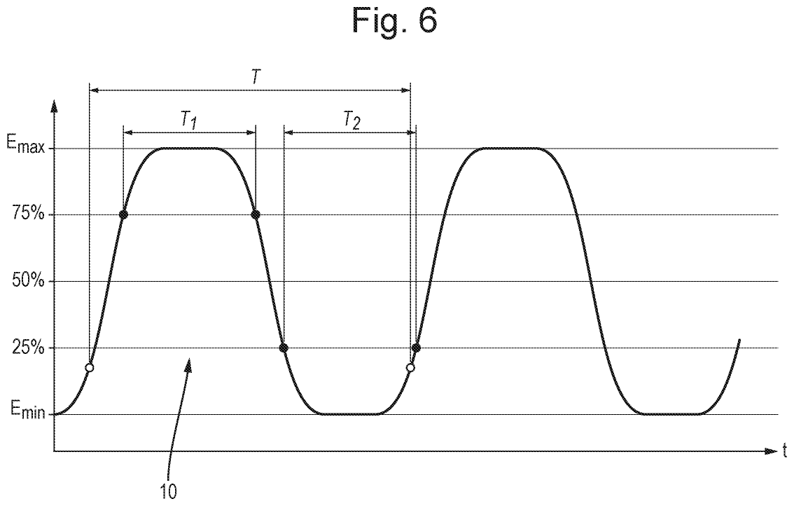

FIG. 6 illustrates a further alternative alternating waveform for modulation of the rate of electrical energy delivered;

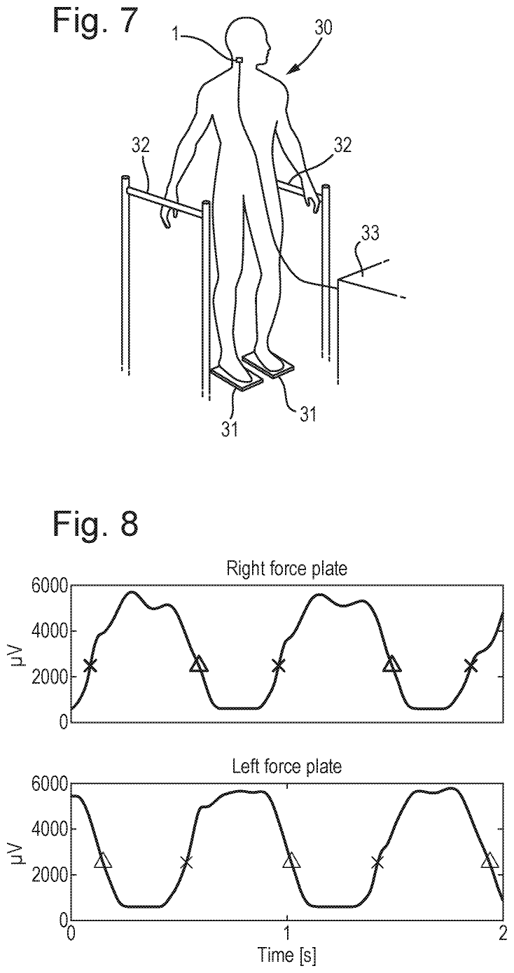

FIG. 7 shows the recording setup used to obtain experimental results discussed herein;

FIG. 8 shows force measurements taken during a step cycle of a patient;

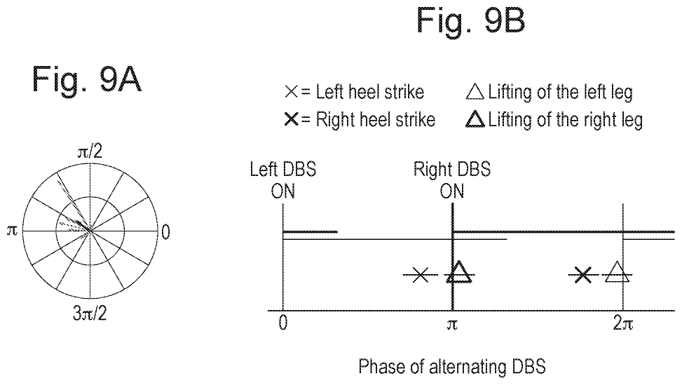

FIGS. 9A and 9B show average entrainment of the gait cycle when altDBS is applied for a group of patients;

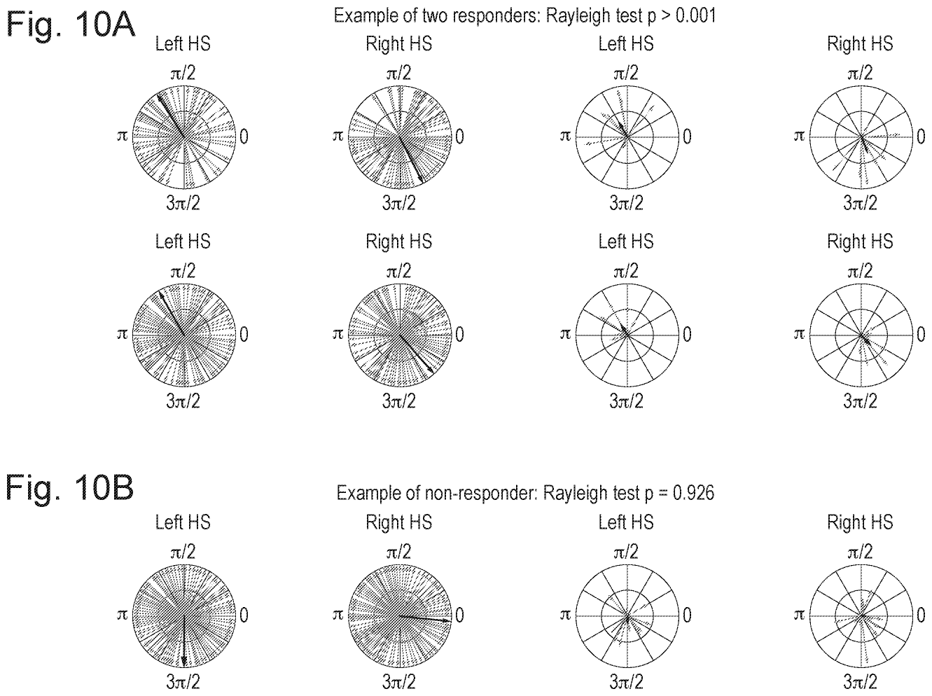

FIGS. 10A and 10B show the differing effects of DBS on patients who responded to DBS, and a patient who did not respond to DBS; and

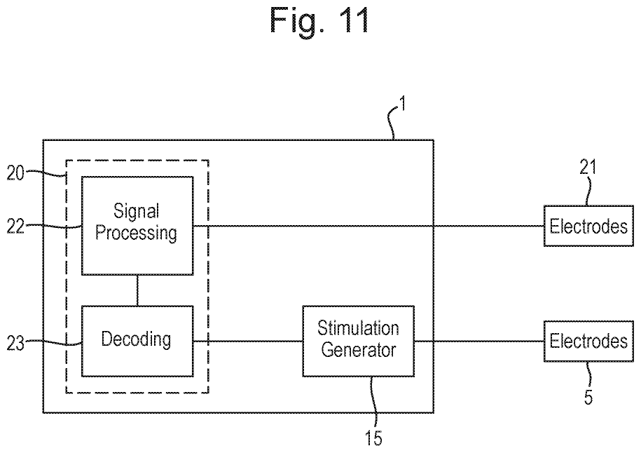

FIG. 11 is a diagram of a DBS device incorporating a tracking system.

The present invention provides a stimulation device 1 for treatment of gait impairment of a patient 2. As discussed above, gait impairment is often caused by Parkinson's Disease. The embodiment of the stimulation device 1 discussed herein is used for the treatment of Parkinson's Disease, and the patient 2 is a patient with Parkinson's Disease. However, in general, other embodiments of the stimulation device 1 may be used to treat patients with other diseases, including but not limited to Progressive Supranuclear Palsy or Multiple System Atrophy.

FIG. 1 shows the stimulation device 1 implanted in the brain 8 of a patient 2. The stimulation device 1 is configured to apply respective stimulation signals 11 to electrodes 5 bilaterally implanted in two subcortical regions 7 of the left and right hemispheres 18 of the brain 8 of the patient 2, the subcortical regions 7 being associated with motor control.

The stimulation device 1 includes a stimulation generator 15 that generates the stimulation signals 11. The stimulation device 1 supplies the stimulation signals to electrodes 5 for bilateral implantation in the two subcortical regions 7 of the left and right hemispheres 18 of the brain 8 of the patient 2. As shown in FIG. 2, the electrodes 5 comprise a first electrode 5a implanted in a subcortical region 7a of a first hemisphere 18a of the brain 8, and a second electrode 5b implanted in a subcortical region 7b of a second hemisphere 18b of the brain 8. The respective stimulation signals 11 are applied to the first electrode and the second electrode respectively. In other words, a first stimulation signal is applied to the first electrode, and a second stimulation signal is applied to the second electrode. The stimulation device 1 is connected by a lead wire 4 to the electrodes 5, which are formed on the tip 6 of the lead wire 4.

The stimulation device 1 may comprises the electrodes 5, but in general it is not necessary that the stimulation device 1 comprise the electrodes 5 and lead wire 4. Implantation of electrodes 5 for DBS is known, and many patients 2 have already undergone surgery to have electrodes 5 implanted. In some embodiments, the stimulation device 1 may be configured to apply respective stimulation signals 11 to electrodes 5 that have previously been implanted in the brain 8 of the patient 2. The stimulation device 1 may be connected to the electrodes 5 via a physical connection such as the lead wires 4, or may transmit the stimulation signals 11 wirelessly to be picked up by the electrodes 5.

Each of the electrodes 5 comprises multiple sub-electrodes electrically connected in parallel. Four sub-electrodes are shown in FIG. 1 by way of example, although any number of electrodes may be used, for example, only one sub-electrode, two sub-electrodes, three sub-electrodes or more than four sub-electrodes.

The subcortical regions 7 are the subthalamic nuclei (STN) for treatment of Parkinson's disease (PD. The subcortical regions 7 may alternatively be the pedunculopontine nuclei (PPN). In general the subcortical regions 7 could be other sites in the brain 8 depending on the disease to be treated. The stimulation device 1 is implanted into the thorax 9 of the patient 2 near the collarbone, with the lead wire 4 extending under the skin of the patient 2. However, in some embodiments, the stimulation device 1 may be external to the body of the patient 2, for example when the stimulation signals 11 are transmitted to the electrodes 5 wirelessly.

The stimulation device 1 is configured to apply respective stimulation signals 11. As shown in FIG. 3, the stimulation device 1 generates a stimulation signal 11 comprising stimulation pulses 12, 13. The stimulation signal 11 has a stimulation frequency Fₛ=1/Tₛ, where Tₛ is the time period of the stimulation signal 11, i.e. the length in time of the repeating cycle of the stimulation signal 11. As shown in FIG. 3, the time period Tₛ is the time between equivalent points on consecutive stimulation pulses 12, 13 of the stimulation signal 11.

The stimulation frequency Fₛ may vary from patient to patient and/or vary for different stimulation target areas. In some embodiments, the stimulation signals 11 have a stimulation frequency Fₛ of at least 20 Hz, preferably at least 50 Hz. In some embodiments, the stimulation signals 11 have a stimulation frequency Fₛ of at most 180 Hz. For example, the stimulation signals 11 may have a stimulation frequency Fₛ of 80 Hz, 100 Hz, or 130 Hz.

When the decision to implant electrodes 5 for continuous DBS of a patient 2 is made, a clinician will commonly determine an appropriate stimulation frequency Fₛ for a continuous clinical stimulation signal. In some embodiments, the stimulation signals 11 have a stimulation frequency Fₛ that is at least 20% of the frequency of a clinical stimulation signal that is clinically defined for use during continuous chronic stimulation, preferably at least 50%, more preferably at least 80%. In some embodiments, the stimulation signals 11 have a stimulation frequency Fₛ that is at most 140% of the frequency of a clinical stimulation signal that is clinically defined for use during continuous chronic stimulation, preferably at most 120%.

In FIG. 3, each cycle of the stimulation signal 11 comprises two stimulation pulses 12, 13 with respectively positive and negative amplitude. Having each cycle of the stimulation signal 11 comprise two stimulation pulses 12, 13 of opposite sign helps to prevent charge build-up in the brain 8 of the patient 2, which can be desirable in some situations. However, this is not essential, and the stimulation signal 11 may instead comprise stimulation pulses 12, 13 all having positive voltage, or all having negative voltage. Each cycle of the stimulation signal 11 may also comprise a number of stimulation pulses 12, 13 other than two. For example, only a single stimulation pulse may be used, or more than two stimulation pulses. Where it is desirable to reduce charge build-up, an even number of stimulation pulses is preferred, wherein half of the stimulation pulses 12, 13 have a positive amplitude, and the other half have negative amplitude.

As shown in FIG. 3, the first stimulation pulse 12 has a duration W1, and the second stimulation pulse 13 has a duration W2. The two stimulation pulses 12, 13 are separated by a period of zero voltage with a duration Wi. The two stimulation pulses 12, 13 have equal and opposite amplitude with a magnitude of A. The pulse width W of the stimulation signal 11 is defined as the time during each cycle of the stimulation signal 11 for which a non-zero voltage is applied. This will be the sum of the durations of all of the stimulation pulses 12, 13 within one cycle of the stimulation signal 11. In FIG. 3, W=W1+W2. The amplitude A of the stimulation signal 11 is the magnitude of the stimulation pulses 12, 13 within each cycle of the stimulation signal 11, i.e. A in FIG. 3.

During each cycle of the stimulation signal 11, the stimulation signal 11 delivers an amount of electrical energy Eᵢ to each electrode 5. Eᵢ is given by:

$${E}_{i} = \frac{{A}^{2}}{R} W$$

where R is the measured system impedance. The rate of electrical energy delivered (REED) by the stimulation signal 11 is therefore:

$$REED = \frac{{E}_{i}}{{T}_{s}} = {E}_{i} {F}_{s} = \frac{{A}^{2}}{R} {WF}_{s}$$

As mentioned above, when continuous DBS is prescribed, a clinical stimulation signal will be clinically defined for use during continuous chronic stimulation. This clinical stimulation signal will have a REED that can be determined using the formula above. Typically, variation in the stimulation frequency Fₛ is used to vary the REED for a clinical stimulation signal. However, the REED can also be altered by varying the amplitude A, or pulse width W of the stimulation signal 11.

In the present invention, the stimulation signals 11 have a rate of electrical energy delivered (REED) to respective ones of the electrodes 5 bilaterally implanted in the two subcortical regions 7 that is modulated with alternating waveforms 10 at a gait frequency and out of phase with each other. In other words, a first alternating waveform used to modulate the rate of electrical energy applied by the first stimulation signal to the first electrode implanted in a subcortical region 7 of the first hemisphere of the brain 8 is out of phase with a second alternating waveform used to modulate the rate of electrical energy applied by the second stimulation signal to the second electrode implanted in a subcortical region 7 of the second hemisphere of the brain 8. This form of modulated DBS is referred to as alternating deep brain stimulation (altDBS). The inventors have found that results such as those given in the examples section below raise the possibility that alternating stimulation of the STN could provide a novel DB S approach to the treatment of gait dysfunction in PD, by reinforcing the normal stepping cycle.

FIG. 4 demonstrates the rate of electrical energy delivered to each hemisphere of the brain 8 in the example experiment discussed further below. In FIG. 4, the alternating waveforms 10 are square waveforms, with a 66% duty cycle. The REED varies between 100% and 0% of its maximum value. The alternating waveforms 10 are out of phase by 50% of the period of the alternating waveforms 10. That is, the first alternating waveform and the second alternating waveform have the same period, and have a phase difference of 50% or π radians. These were the parameters chosen for the experiment discussed below, but neither of these features are essential.

The alternating waveforms 10 may in general comprise any shape of waveform, for example triangular waves, sawtooth waves, sinusoidal waves, or other arbitrary waveforms. Similarly, although the alternating waveforms 10 in FIG. 4 are out of phase by 50% of the period of the alternating waveforms 10, this is also not essential. A 50% phase difference may be preferred as reflecting a typical phase difference between movements of each foot of the patient 2 during a normal step cycle. However, variations in the phase difference may be made, for example depending on the gait pattern of a particular individual. In an embodiment, the phase difference between the two alternating waveforms 10 is between 40% and 60%.

As discussed above, the rate of electrical energy applied is determined by the stimulation frequency, the amplitude of the stimulation signal 11, and the pulse width of the stimulation signal 11. Any of these parameters may be used to control the rate of electrical energy delivered. In an embodiment, the stimulation device 1 is configured to apply respective stimulation signals 11 having the rate of electrical energy delivered modulated by modulation of an amplitude of the stimulation signals 11. In an embodiment, the stimulation device 1 is configured to apply respective stimulation signals 11 having the rate of electrical energy delivered modulated by modulation of a stimulation frequency of the stimulation signals 11. In an embodiment, the stimulation device 1 is configured to apply respective stimulation signals 11 having the rate of electrical energy delivered that is modulated by modulation of a pulse width of the stimulation signals 11. The alternating waveform 10 may be used to determine the modulation of any of these parameters. It is also possible to control more than one of these parameters simultaneously to vary the REED. The stimulation device 1 may be configured to apply respective stimulation signals 11 having the rate of electrical energy delivered modulated by modulation of one or more of: an amplitude of the stimulation signals 11; a stimulation frequency of the stimulation signals 11; and a pulse width of the stimulation signals 11. In an embodiment, the rate of electrical energy delivered may be modulated by modulation of two of these parameters, or optionally all three of these parameters.

In FIG. 4 (and similarly FIG. 5 below), it is assumed that the respective stimulation signals 11 are modulated with identical alternating waveforms 10. In other words, the first waveform 10 modulating the stimulation signal 11 applied to the first electrode is identical to the second waveform 10 modulating the stimulation signal 11 applied to the second electrode, apart from the phase difference between the first waveform and the second waveform. However, while this may be preferred for clinical reasons, it is not essential. In some situations, it may be desirable to apply a stimulation signal 11 with, for example, a higher maximum rate of electrical energy delivered to one of the electrodes 5 than to the other of the electrodes 5. This may be, for example, if there are differences in structure between the two hemispheres of the patient's brain 8.

FIG. 5 shows an alternative example of an alternating waveform 10 used to modulate the rate of electrical energy delivered to the electrodes 5. The alternating waveform 10 shown in FIG. 5 is also a square wave, similar to the alternating waveforms 10 of FIG. 4. However, the alternating waveform 10 in FIG. 5 has a duty cycle of 50%, and does not vary in amplitude between zero and its maximum. The alternating waveform 10 of FIG. 5 varies between a maximum value Eₘₐₓ and a minimum value Eₘᵢₙ. Therefore, a stimulation signal 11 with a non-zero rate of electrical energy delivered is applied to the electrodes 5 at all times, but the stimulation signal 11 has a different stimulation frequency, amplitude, or pulse width during different parts of the cycle.

In order to provide the desired effect on gait impairment, there must be a clear definition of the change in the parameters of the stimulation signal 11 caused by the alternating waveform 10. In an embodiment, the alternating waveforms 10 have a maximum rate of electrical energy delivered Eₘₐₓ and a minimum rate of electrical energy delivered Eₘᵢₙ, the minimum rate of electrical energy delivered Eₘᵢₙ being 65% or less of the maximum rate of electrical energy delivered Eₘₐₓ, preferably 50% or less, more preferably 25% or less.

Modulating the rate of electrical energy delivered may mean that the total electrical energy delivered over several cycles of the alternating waveform 10 is lower than the total electrical energy that would be delivered over the same time period by a corresponding continuous deep brain stimulation system. This may affect the effectiveness of the altDBS in controlling the symptoms of gait impairment. For this reason, in some embodiments, the alternating waveforms 10 have a maximum rate of electrical energy delivered Eₘₐₓ that is at least 100% of the rate of electrical energy delivered of a clinical stimulation signal that is clinically defined for use during continuous chronic stimulation, optionally at least 120%. However, this is not essential, and effective relief of symptoms may still be achieved even if the maximum rate of electrical energy delivered Eₘₐₓ is below the rate of electrical energy delivered of a clinical stimulation signal for continuous DBS.

Having too high a rate of electrical energy delivered may also create undesirable side effects in the patient's brain 8. Therefore, in an embodiment, the alternating waveforms 10 have a maximum rate of electrical energy delivered Eₘₐₓ that is at most 200% of the rate of electrical energy delivered of a clinical stimulation signal that is clinically defined for use during continuous chronic stimulation, optionally at most 150%. In order to provide clear contrast between the maximum Eₘₐₓ and minimum Eₘᵢₙ of the alternating waveform 10, in some embodiments the alternating waveforms 10 have a minimum rate of electrical energy delivered Eₘᵢₙ that is at most 65% of the rate of electrical energy delivered of a clinical stimulation signal that is clinically defined for use during continuous chronic stimulation, optionally at most 50%.

In some embodiments, it may be desirable to use more complex shapes of alternating waveform 10 than those shown in FIG. 4 and FIG. 5. FIG. 6 shows an example of such an alternating waveform 10. The period of the alternating waveform 10 is T. The alternating waveform 10 in FIG. 6 varies between a maximum value Eₘₐₓ and a non-zero minimum value Eₘᵢₙ, similar to the alternating waveform 10 of FIG. 5. For more complex alternating waveforms 10 such as the example in FIG. 6, it is still necessary to provide clear contrast between the periods having a lower rate of electrical energy delivered, and periods having a higher rate of electrical energy delivered.

In an embodiment, the rate of electrical energy delivered of the alternating waveforms 10 remains in the upper quartile of the range between the minimum rate of electrical energy delivered Eₘᵢₙ and maximum rate of electrical energy delivered Eₘₐₓ for at least 10% of the period of the alternating waveforms 10, preferably for at least 20% of the period of the alternating waveforms 10. The period for which the rate of electrical energy delivered remains in said upper quartile is shown as T₁ in FIG. 6. In some embodiments, the rate of electrical energy delivered of the alternating waveforms 10 remains in the upper quartile of the range between the minimum rate of electrical energy delivered Eₘᵢₙ and maximum rate of electrical energy delivered Eₘₐₓ for at most 90% of the period of the alternating waveforms 10, preferably for at most 70% of the period of the alternating waveforms 10. In FIG. 6, the rate of electrical energy delivered remains in the upper quartile for approximately 40% of the period of the alternating waveforms 10.

Similarly, in some embodiments, the rate of electrical energy delivered of the alternating waveforms 10 remains in the lower quartile of the range between the minimum rate of electrical energy delivered Eₘᵢₙ and maximum rate of electrical energy delivered Eₘₐₓ for at most 90% of the period of the alternating waveforms 10, preferably for at most 80% of the period of the alternating waveforms 10. The period for which the rate of electrical energy delivered of the alternating waveforms 10 remains in said lower quartile is shown as T₂ in FIG. 6. In some embodiments, the rate of electrical energy delivered of the alternating waveforms 10 remains in the lower quartile of the range between the minimum rate of electrical energy delivered Eₘᵢₙ and maximum rate of electrical energy delivered Eₘₐₓ for at least 10% of the period of the alternating waveforms 10, preferably for at least 30% of the period of the alternating waveforms 10. In FIG. 6, the rate of electrical energy delivered remains in the lower quartile for approximately 40% of the period of the alternating waveforms 10. Note that, because the alternating waveform 10 in FIG. 6 is not a square waveform, T₁+T₂<T.

The time period T of the alternating waveform 10 defines the gait frequency at which the stimulation signals 11 are modulated. In an embodiment, the gait frequency is a desired gait frequency. The desired gait frequency may be determined in a number of different ways. In an embodiment, the desired gait frequency is a gait frequency measured in the absence of stimulation. Alternatively, the desired gait frequency may be a gait frequency measured while the patient 2 is receiving continuous deep brain stimulation, for example in accordance with a previously-prescribed continuous DBS treatment. As a further alternative, the desired gait frequency may reflect a given individual's preferred walking speed. In some embodiments, the gait frequency may be adjusted during altDBS by detecting the gait frequency of the patient 2. In some situations, it may not be practical to determine the natural or preferred gait frequency of an individual patient. Therefore, in some embodiments, the desired gait frequency is in the physiological range for an age group of the patient 2. It is possible to prompt a patient 2 to carry out a faster stepping rhythm.

The stimulation device 1 may also be used to implement a corresponding method of treatment of gait impairment of a patient 2. The method comprises applying respective stimulation signals 11 to electrodes 5 bilaterally implanted in two subcortical regions 7 of the left and right hemispheres of the brain 8 of the patient 2. The subcortical regions 7 are regions associated with motor control. The respective stimulation signals 11 are amplitude modulated by alternating waveforms 10 at a gait frequency and out of phase with each other. The alternating waveforms 10 and stimulation signals 11 used in the method may be as described above for the stimulation device 1.

### Examples

There will now be described an exemplary embodiment of the stimulation device 1 described above, and an experiment demonstrating the results of using the stimulation device 1 to treat patients with Parkinson's Disease.

### Introduction

In this study, alternating DBS was delivered in cycles which matched the duration of the stepping cycle or were 20% shorter. Stimulation intensity was at the clinically effective voltage for two thirds of the stimulation cycle and was briefly lowered to 0V in most cases for one third of the stimulation cycle (FIG. 4). This rhythm was provided with an offset between the left and right STN such that the pauses occurred at opposite points within one full stimulation cycle. Our primary objective was thus to find out if alternating STN DBS can entrain the step cycle to the DBS pattern.

We therefore tested alternating deep brain stimulation during stepping in place in 10 patients with Parkinson's disease and chronically implanted subthalamic nuclei (1 female, age 50-73 years). In the first condition, stimulation was delivered in cycles of similar duration to each patient's preferred stepping cycle. We stimulated at the clinically effective voltage for two thirds of the stimulation cycle and lowered stimulation intensity to as low as 0V for the remaining time. This pattern was offset between the left and right subthalamic nucleus by half a stimulation cycle such that the high- and low-intensity periods were delivered in an alternating pattern. Alternating DBS significantly entrained patients' stepping rhythms at the group level (p=0.002), eliciting a consistent alignment between the step cycle and the stimulation cycle.

General motor symptoms, assessed with a double-blind UPDRS-III (Unified Parkinson's Disease Rating Scale III) examination, did not significantly differ between alternating deep brain stimulation and conventional continuous stimulation (p=0.254). Our study provides evidence that the subthalamic nucleus is causally involved in the dynamic control of stepping. The inventors predict that this novel deep brain stimulation pattern can provide the basis for improved closed-loop stimulation strategies to ameliorate gait impairments.

### Materials and Methods

We recorded 10 Parkinson's patients (mean age 67±(STD) 7 years, disease duration 14.2±4 years, time since DBS implantation 3.8±1.3 years, 1 female) with chronically implanted STN DBS electrodes, who had received DBS surgery 1-5 years previously at University College London Hospital (UCLH) in London (n=9) or at the Hadassah Hospital in Jerusalem, Israel (n=1).

All patients were implanted with the Medtronic Activa-PC neurostimulator and the 3389 macroelectrode model to alleviate their motor symptoms. We considered patients younger than 80 years for this study. None of the participants had cognitive impairments, which were assessed with a mini mental score examination (≥26/30 see Table 1). Interleaved stimulation as a DBS setting was an exclusion criterion because the streaming telemetry system Nexus-D (Medtronic, USA) that was used to control alternating stimulation cannot deliver interleaved stimulation.

Our main objective for this study was to find out if participants would entrain to the alternating DBS pattern and how their step timing would align to the stimulation pattern. Therefore, we did not specifically recruit patients with severe gait impairments but also included patients that experienced no gait impairments such as freezing or festination. Patients' severity of gait impairments was assessed at the beginning of their visit with a gait and falls questionnaire.

### Stimulation Conditions and Setting the DBS Parameters

All patients performed stepping in place while standing during three stimulation conditions: Conventional continuous DBS, alternating DBS at their preferred stepping speed and alternating DBS 20% faster than their preferred speed. We will refer to the latter as fast alternating DBS in the following sections. Some patients also performed the stepping movement when stimulation was switched off (n=5), but because time constraints allowed this only in half of all patients this condition was not further analysed. All recordings were performed on medication to limit fatigue. Before changing DBS to the alternating pattern, patients' preferred stepping speed was measured during ˜30 s free walking and during ˜20 s stepping in place (while DBS was on continuously) with a MATLAB script that registered the time interval between key presses performed by the experimenter at the patient's heel strikes. The median interstep interval was used to determine the duration of the stimulation cycles in the two alternating DBS conditions during stepping in place. The preferred duration of one full gait cycle was 1.2 s in most cases (stepping in place: mean=1.27±0.22 s, ranging between 1.1-1.8 s, free walking: mean=1.18±0.17 s, ranging between 0.94-1.4 s). There was no significant difference between the two conditions (t₆=0.5, p=0.664; df=6 because the preferred speed of free walking was only measured in the final six patients). The median interstep interval from the stepping in place measurement was used to determine the duration of the stimulation cycles in the two alternating DBS conditions during stepping in place. The stimulation intensity and timing delivered by the chronically implanted pulse generator were remotely controlled by the Nexus-D device, which communicated via telemetry. The stimulation intensity was at the clinically effective voltage for two thirds of the stimulation cycle and was lowered intermittently only for one third of the full stimulation cycle (as shown in FIG. 4). This rhythm was provided with an offset between the left and right STN such that the pauses occurred at opposite points within one full stimulation cycle. This 67/33% pattern was chosen because the technical limitations of Nexus-D would have not allowed a 50/50% pattern as the device requires gaps of at least 100 ms to reliably send two consecutive commands (left up, right down, right up, left down, see FIG. 4). We opted for 67% instead of 33% for the high-intensity stimulation period to keep the overall stimulation intensity relatively high in comparison to continuous DBS.

A typical alternating stimulation cycle thus consisted of 0.8 s (=⅔ of 1.2 s) of standard intensity stimulation (drawn from the clinically effective voltage during chronic continuous stimulation) and 0.4 s (=⅓ of 1.2 s) of lowered intensity or no stimulation. FIG. 4 shows the alternating DBS pattern. DBS was set to the clinically effective voltage for ⅔ of the stimulation cycle and reduced for ⅓ of the cycle. For the reduced period, stimulation intensity was set to 0V in eight patients and it was reduced by −1V and −1.2V relative to the clinically effective threshold in the remaining two. The pattern was offset between the left and right STN such that the pauses occurred at exactly opposite points of the stimulation cycle. Grey dashed lines show the start and end of one full stimulation cycle (compare with FIG. 9B).

The lower limit of alternating stimulation was determined by reducing the clinically effective voltage in steps of −0.5V and evaluating if the patient noticed a change until reaching 0V. If troublesome symptoms appeared before reaching 0V, the lower limit remained above the side effects threshold. In 8 of 10 patients the lower limit was set to 0V with patients reporting that alternating stimulation was well tolerated. In one patient (P06), reducing the lower limit by more than 1.2V resulted in reappearance of tremor and in another patient (P10) it caused headache at the forehead and slight tingling of the lips, which immediately disappeared when stimulation was switched back to the continuous mode. These two patients were the only participants with an upper stimulation threshold (based on their clinical stimulation settings) that differed between the left and the right STN (see P06 and P10 in Table 1). Their lower limits were set separately for the left and right STN to −1V (P06) and −1.2V (P10) below the upper thresholds, so that the patients were spared tremor and tingling. Other minor side effects in other patients were slight dizziness in one case and increased clarity, ‘as if a fog has been lifted’, in another case. Patients were informed of each change in stimulation intensity whilst the lower threshold for stimulation was sought.

Note that before using Nexus-D to switch to the alternating stimulation mode, the amplitude limits of the patient programmer option in the stimulator were adjusted with Medtronic NVision: We set the upper limit to ‘+0V’ relative to the clinical amplitude (drawn from the clinically effective voltage during chronic continuous stimulation) and the lower limit to ‘-clinical amplitude’ to ensure that the stimulation amplitude could never be increased above the clinically effective amplitude.

### Task

FIG. 7 shows the recording setup. Patients 30 with a stimulation device 1 performed stepping in place on force plates 31 (Biometrics Ltd ForcePlates) at their comfortable speed and maintain a consistent movement throughout the recording. Two parallel bars 32 were placed to the left and right of the force plates 31 to allow patients 30 to hold on to them if they wanted more stability or if they felt more comfortable resting their arms on the bars. Output from the force plates 31 was supplied to a recording system 33

The experimenter asked patients to ‘Start stepping whenever you are ready’. After about 20 s they were prompted to stop and pause. For the first three patients the prompt was given verbally, and for the subsequent patients a mobile phone countdown triggered an auditory alarm after 20 s to prompt the pause.

The duration of the pauses was randomly varied (the shortest pause was 2.7 s) and they could extend up to several minutes as patients were allowed to sit down and rest between the 20 s sequences whenever they wanted. To control for any effects of fatigue that may increase with time, we recorded the three conditions (continuous DBS, alternating DBS and fast alternating DBS) in the following order: A B C C B A, with 5-6 sequences in each block (except in one patient, P05, who completed only A B C as he was too tired to complete the full set). The order of the stimulation conditions was balanced across patients, so that A would in turn refer to continuous DBS, alternating DBS or fast alternating DBS. The stimulation was set to one mode for the whole duration of each experimental block without any pauses or resets between stepping sequences or rest intervals.

Patients were not told what stimulation condition was active. They also did not report any conscious rhythmic sensations and thus could not discern the rhythm of the alternating stimulation. The experimenter controlled the stimulation modes using custom-written software and was thus aware of the stimulation conditions but was unaware of the precise timing of the stimulation onset when prompting patients to start stepping any time again.

Either before or after the stepping task, a blinded clinical research fellow performed the UPDRS-III motor examination (on medication), once during continuous DBS and once during alternating DBS. The order was randomized across patients so that continuous DBS was the first condition for half of all patients. Stepping in place provides only a proxy measure of stereotypical gait, but as part of the clinical examination a 20 m free walking assessment was also performed in a corridor. For the first patients, Bluetooth communication was not yet available and one experimenter had to walk next to the patient carrying the laptop connected via USB with the Nexus-D. For the final six patients, Bluetooth communication between the laptop and Nexus-D allowed the patients to walk freely during both alternating DBS and continuous DBS. Alternating DBS was set to the individual's preferred speed that was recorded during free walking. In these six patients, we also measured the time and number of steps needed to complete a 10 m straight walk, turn and return to the starting point. Note that the step timing relative to stimulation was not recorded during free walking, and thus the strength of entrainment could not be assessed. The complete visit lasted up to 2.5 hours including extended pauses between individual assessments.

### Recordings

The recording system 33 was arranged as follows. A TMSi Porti amplifier (2048 Hz sampling rate, TMS International, Netherlands) recorded continuous force measurements from the two force plates, which were taped to the floor, to extract the step timing. Triggers indicating the onsets of high-intensity stimulation were recorded with a light-sensitive sensor attached to the screen of the laptop that controlled stimulation timing via the Nexus-D. The screen below the sensor displayed a grey box that briefly turned black at the onset of high-intensity stimulation in the left electrode and white for the onset in the right electrode. DBS artefacts that captured if stimulation was on, and in which mode, were recorded with two bipolar electrodes attached to the back of the neck slightly below the ears. This measurement provided a simple check during the experiment that allowed us to see if the stimulation protocol was working.

### Data Processing

Heel strikes were identified in Spike2 (Cambridge Electronic Design Limited) based on the force measurements by setting a threshold for each patient to capture approximately the midpoint of each force increase. FIG. 8 shows the force measurements and step cycle events. x=heel strikes. The force increased during heel strikes. Δ=when the foot was raised from the force plate and the force decreased.

The force measurement increased whenever weight was transferred onto a force plate. Note that the foot touched the force plate already slightly earlier, about 100 ms before, the heel strike event, however, considerable weight was only transferred on the leg by the time of the event. We used the same threshold for identifying when the leg was lifted, which was captured by a force decrease. Note here again that the foot was fully lifted off the plate only slightly after the event, however, the process of lifting the leg up was initiated already before then.

To avoid biasing the entrainment results by sequences that were several seconds longer than other sequences, which occurred occasionally when verbal prompts were used to prompt stopping, steps at the beginning and end of the longer sequences were removed, such that the remaining number of steps did not exceed the median number of steps of all the sequences. Freezing episodes were very rare and were excluded from the analyses. They occurred in two patients (P03, P04) towards the end of the recording session without any apparent difference between conditions.

### Statistical Analysis

All analyses were performed with MATLAB (v. 2016a, The MathWorks Inc., Natick, Mass.). Entrainment of the step timing to the stimulation pattern was evaluated with a Rayleigh-test (using the MATLAB toolbox CircStat; Berens, 2009) for each individual patient and with a permutation procedure at the group level. Here we define entrainment as significant alignment of the timing of steps to the rhythm of the alternating stimulation pattern. This alignment was evaluated with a Rayleigh-test (using the MATLAB toolbox CircStat; Berens, 2009) for each individual patient and with a permutation procedure at the group level that considers each individual's average timing and entrainment strength. Whenever a heel strike occurred (tests are only reported for the left heel strikes, because p-values were highly similar for the right heel strike), the coincident phase of the rhythmic alternating DBS pattern was extracted. The uniformity of this resulting phase distribution was then assessed with a Rayleigh-test to test if individual patients showed significant entrainment. An additional permutation procedure was used to compute a group statistic across all ten recorded patients. For the group statistic, the vector length was calculated first for each patient according to the formula

$$\vert \frac{{\sum }_{s = 1}^{N} {e}^{i * {ϕ}_{s}}}{N} \vert$$

where ϕₛ is the phase of alternating DBS at each left heel strike and N the number of all heel strikes.

The grey dashed lines in FIG. 4 show the start and end of one full stimulation cycle, and the x-axis in FIG. 9B shows the phase of one alternating stimulation cycle. Note that whenever we show arrows representing phases, they always refer to the phase of alternating stimulation at the time of the patients' heel strikes and not to the phase of their stepping cycle, which was another cyclic measurement. The circular mean of these phases was then computed to obtain the average ‘preferred’ phase for each patient. This resulted in ten vectors (one for each patient) with their direction representing the average preferred phase, and their length representing the strength of entrainment (grey vectors in FIG. 9A). Next, they were transformed into Cartesian coordinates and the average of the ten vectors (black vector in FIG. 9A) was computed. The length of this average vector was obtained using Pythagoras' theorem and was our group statistic of interest. It takes into account both the strength of entrainment and the consistency of the preferred phases across patients.

FIG. 9 shows the results of entrainment at the group level. In FIG. 9A, the grey vectors show the average phase of alternating DBS at all left heel strikes and the strength of entrainment for individual patients (n=10). Long arrows show strong entrainment. The group average vector (black vector) shows the average of the grey vectors. The length of this vector was significantly larger than in the surrogate data, demonstrating consistent alignment of stepping to the alternating DBS pattern across the group. FIG. 9B shows group-averaged timing of key events of the gait cycle (x and Δ) relative to the stimulation pattern, with horizontal bars indicating the standard error of the mean phases across the patients. The normal and bold horizontal lines indicate high-intensity stimulation of the left and right STN, respectively. As shown, the left heel strike was made just before contralateral stimulation increased.

If all patients would have shown strong entrainment, but with different preferred phases, the length of the group average vector would be close to zero. Only if the vectors representing individual patients pointed into a similar direction, the group average vector would be significantly larger than the one obtained from our permutation data.

We computed a permutation distribution of 1000 surrogate vector lengths by shifting, separately for each patient, each of their 20 s long stepping sequences in time by a random offset drawn from a uniform distribution ranging between −1.5 s and +1.5 s. This way the rhythmic structure within the 20 s stepping sequences remained intact and only their relative alignment to the stimulation pattern was randomly shifted. Once all sequences were randomly shifted, we computed the surrogate vector length and preferred phase for each patient as described above for the unpermuted data. The resulting ten surrogate vectors were again averaged in the Cartesian coordinate system to compute the average length as described above. After repeating this 1000 times, we obtained a p-value by counting how many of the surrogate group vector lengths (Lₚ) were larger or equal to the original group vector length (L_{orig}) and dividing this number by the number of permutations (Nₚ). The number 1 is added to both the nominator and the denominator to avoid p-values of 0 and be consistent with the exact p-value, which must be at least 1/Nₚ (see section 4.2 from Ernst, 2004):

$${p}_{value} = \frac{1 + {\sum }_{p = 1}^{{N}_{p}} f ( {L}_{p} )}{1 + {N}_{p}} ,$$

$$f ( {L}_{p} ) = \{ \begin{matrix}0 , & {L}_{p} < {L}_{orig} \\ 1 , & {L}_{p} \geq {L}_{orig}\end{matrix}$$

As we expected entrainment to be strongest when the stimulation speed matches the patient's stepping speed as closely as possible, the group statistic was based on the data from the alternating DBS condition that matched the patient's stepping speed most closely.

All patients that showed significant entrainment indeed did so in the condition that was closest to their stepping speed. The stepping pace of several patients (P03-P08) was considerably faster during the recording than in the brief initial assessment, hence in those, the fast alternating DBS condition matched their performed stepping rhythm more closely.

Pairwise comparisons of the step intervals between the two alternating DBS conditions and of the change in variability between speed-matched alternating DBS and continuous DBS were performed using two-tailed t-tests or Wilcoxon signed-rank tests (with an alpha-level of 0.05) if the normality assumption (assessed by Lilliefors tests) was violated. To get a robust estimate for each patient and condition, first the median of all step intervals within each 20 s stepping sequence swas computed, and then again the median over all sequences was computed. To investigate the step timing variability, we computed the coefficient of variation of the step intervals (STD/mean*100) as well as the standard deviation of the difference between two consecutive step intervals for each sequence. The median over all sequences was again computed to get a robust estimate. To test in each patient individually if the step timing variability was significantly modulated by alternating DBS, we computed two-samples t-tests or rank-sum tests (if the normality or variance homogeneity assumption was violated) between the step timing variability estimates of the stepping sequences that were recorded in each DBS condition.

### Localization of the Active Electrode Contacts

Each DBS lead has four contacts of which only one or two are activated during stimulation. The location of the active contacts was assessed in Brainlab (Brainlab AG, Germany) by a neurosurgeon and a neurologist who manually drew the lead on the post-operative T1 MR images centred on the DBS electrode artefact. The position of the contacts within the STN was then assessed visually in the patients' pre-operative artefact-free T2 images. We did not have access to imaging data for P7 who received the surgery in Israel, and the quality of the imaging data was insufficient in two patients, so in these cases no accurate estimate of the contact position could be obtained.

Table 1 shows the clinical details, including location of the electrode contact used for stimulation, and stimulation parameters for all patients. Patients who were significantly entrained to alternating DBS are highlighted in bold. No distinct differences between the group of responders and non-responders were apparent with respect to the stimulation intensity boundaries, location of the active contact, severity of motor symptoms or gait problems. The location of the active contacts varied across patients such that some were located in the ventral, some in the dorsal STN, but no pattern emerged that would distinguish between the groups of responders. The only criterion that stood out, and the only parameter that may be associated with entrainment, was the stimulation frequency. The stimulation frequency was either 80 or 100 Hz in the group of responders, but never 130 Hz, which is the conventional frequency for STN DBS. However, note that two non-responders also had a stimulation frequency of 80 and 100 Hz. The four contacts on each electrode are labelled as 0-3 (ventral-dorsal) on the left electrode and 8-11 on the right electrode. The clinically effective stimulation intensity during standard continuous stimulation was set as Upper threshold (rounded to the first decimal place). Stim threshold diff was the difference between the upper threshold and the intensity during the periods of lower or absent stimulation during the alternating mode. This difference was the same in the two sides. All patients received stimulation with a pulse width of 60 μs.

TABLE 1
GFQ = Gait and falls questionnaire. LED = Levodopa equivalent dose.

| | P01 | P02 | P03 | P04 | P05 | P06 | P07 | P08 | P09 | P10 |
|---|---|---|---|---|---|---|---|---|---|---|
| Age | 70 | 71 | 69 | 57 | 73 | 66 | 70 | 69 | 50 | 73 |
| Disease | 19 | 13 | 10 | 18 | 14 | 20 | 9 | 9 | 15 | 15 |
| duration (y) | | | | | | | | | | |
| Months | 64 | 54 | 16 | 42 | 38 | 41 | 69 | 38 | 41 | 52 |
| since | | | | | | | | | | |
| DBS | | | | | | | | | | |
| Preop. | 25 | 29 | 41 | 49 | 33 | 64 | 35 | 92 | 29 | 46 |
| UPDRS | | | | | | | | | | |
| OFF | | | | | | | | | | |
| med | | | | | | | | | | |
| Preop. | 9 | 12 | 11 | 9 | 10 | 22 | 4 | 31 | 11 | 24 |
| UPDRS | | | | | | | | | | |
| ON med | | | | | | | | | | |
| GFQ | 12 | 21 | 34 | 42 | 29 | 13 | 8 | 3 | 15 | 5 |
| Freezing | No | Yes | Yes | Yes | Yes | Yes | No | No | Yes | Not any |
| Yes/No | | | | | | | | | | more |
| Mini- | 29 | 29 | 29 | 28 | 28 | 30 | 27 | 30 | 26 | 28 |
| Mental | | | | | | | | | | |
| Score | | | | | | | | | | |
| LED | 1413 | 384 | 739 | 1223 | 1333 | 645 | 966 | 1169 | 907 | 379 |
| (mg) | | | | | | | | | | |
| Le STN | ventral | N/A | ventral | dorsal | dorsal | dorsal | N/A | dorsal | N/A | midline |
| contact | STN | | STN | STN | STN | STN | | STN | | STN |
| location | | | | | | | | | | |
| Le | 1 | 2 | 1 | 1 | 1 | 2 | 1 + 2 | 1 | 1 | 2 |
| Active | | | | | | | | | | |
| contact | | | | | | | | | | |
| Le | 4 | 2.5 | 3.5 | 2 | 2.5 | 3.5 | 1 | 3 | 1.8 | 2.5 |
| Upper | | | | | | | | | | |
| threshold | | | | | | | | | | |
| (V) | | | | | | | | | | |
| Ri STN | ventral | N/A | ventral | dorsal | dorsal | ventral | N/A | dorsal | N/A | dorsal |
| contact | STN | | STN | STN | STN | + | | STN | | STN |
| location | | | | | | dorsal | | | | |
| | | | | | | STN | | | | |
| Ri | 9 | 9 | 9 | 9 | 9 | 9 + 10 | 9 | 9 | 9 | 9 |
| Active | | | | | | | | | | |
| contact | | | | | | | | | | |
| Ri | 4 | 2.5 | 3.5 | 2 | 2.5 | 2.5 | 1 | 3 | 1.8 | 3.5 |
| Upper | | | | | | | | | | |
| threshold | | | | | | | | | | |
| (V) | | | | | | | | | | |
| Stim. | 80 | 100 | 100 | 100 | 130 | 100 | 170 | 80 | 130 | 80 |
| freq. | | | | | | | | | | |
| (Hz) | | | | | | | | | | |
| Stim. | 4 | 2.5 | 3.5 | 2 | 2.5 | 1 | 1 | 3 | 1.8 | 1.2 |
| threshold | | | | | | | | | | |
| diff. (V) | | | | | | | | | | |

### Results

### UPDRS-III Examination

The blinded UPDRS-III assessment showed no significant differences between continuous DBS (25.1±(STD) 5.7) and alternating DBS (26.5±6.45, Wilcoxon signed-rank test (n=10), p=0.254). The UPDRS items 27-31 reflecting balance and gait also were very similar (in seven patients the scores were identical between conditions, and p-values of the signed-rank tests were 1.0; mean item 27: contDBS=0.8±0.6, altDBS=0.9±0.9; item 28: contDBS=0.8±0.6, altDBS=0.9±0.9; item 29: contDBS=1.2±0.4, altDBS=1.2±0.4; item 30: contDBS=1.0±0.7, altDBS=1.1±0.9; item 31: contDBS=1.4±0.5, altDBS=1.5±0.7). In the six patients that performed the timed 20 m walking assessment (walk 10 m straight, turn and return back to the starting point) the time needed and numbers of steps did not differ significantly between stimulation conditions (continuous DBS: 19.8 s±5.2 s and 35±8 steps, alternating DBS: 19.8 s±4.5 s and 35±6 steps).

### Entrainment to Alternating DBS

Patients started each sequence of 20 s stepping while alternating DBS was already ongoing. Testing for significant entrainment of their steps to the stimulation pattern thus quantifies to which extent patients aligned their stepping rhythm in each sequence to the ongoing stimulation pattern despite not being consciously aware of the precise pattern.

We found significant entrainment of the stepping movement to alternating DBS at the group level based on our surrogate procedure (p=0.002). This highlights that the preferred phase was relatively consistent across patients (FIG. 9A). We also confirmed this finding using a simple Rayleigh test, comparing the preferred phases across patients irrespective of the strength of their entrainment, as this cannot be taken into account by a conventional Rayleigh-test. This demonstrated again significant clustering of three of the four stepping events (left heel strike p=0.109, right heel strike: p=0.033, left leg raised: p=0.020; right leg raised: p=0.015).

On an individual level, five of the ten recorded patients showed significant entrainment in the speed-matched stimulation condition.

Table 2 shows the stimulation speed, stepping speed and p-values testing for significant entrainment in the two alternating DBS conditions. The p-values in bold highlight the patients that were significantly entrained to the alternating DBS pattern (assessed with Rayleigh-tests). Significant entrainment always occurred in the condition where the stepping speed was closer to the stimulation speed. Only P02 was also entrained to alternating DBS in the other condition. P05 and P07 reported that when stimulation was switched off outside of this study, they did not notice an immediate deterioration of symptoms, suggesting that DBS only had weak positive effects. These two patients were not entrained to alternating DBS.

TABLE 2

| | alt DBS | | | fast alt. DBS | | |
|---|---|---|---|---|---|---|
| | stimSpeed | stepSpeed | p-value | stimSpeed | stepSpeed | p-value |
| P01 | 1.2 | 1.12 | 0.317 | 0.96 | 1.07 | 0.079 |
| P02 | 1.8 | 1.69 | <0.001 | 1.44 | 1.62 | 0.039 |
| P03 | 1.2 | 0.91 | 0.893 | 0.96 | 0.91 | <0.001 |
| P04 | 1.2 | 0.91 | 0.845 | 0.96 | 0.81 | 0.744 |
| P05 | 1.1 | 0.87 | 0.124 | 0.88 | 0.77 | 0.976 |
| P06 | 1.2 | 0.92 | 0.762 | 0.96 | 0.91 | 0.007 |
| P07 | 1.1 | 0.98 | 0.875 | 0.88 | 0.77 | 0.738 |
| P08 | 1.2 | 0.86 | 0.878 | 0.96 | 0.84 | 0.008 |
| P09 | 1.5 | 1.39 | 0.841 | 1.2 | 1.41 | 0.728 |
| P10 | 1.2 | 1.22 | <0.001 | 0.96 | 1.14 | 0.994 |

FIG. 10A shows two examples of patients that were significantly entrained (P02 and P03) and FIG. 10B shows one example of a patient that was not entrained. The two plots to the left show the stimulation phases coinciding with the left and right heel strikes. The plots to the right with fewer arrows show the preferred phase and strength of entrainment for each of the separate sequences of 20 s stepping that patients performed. The arrows are clustered again around the preferred phase in the patient that was entrained to the stimulation pattern, which was not the case in FIG. 10B.

In FIG. 10A, grey vectors show the phases of the alternating stimulation pattern at the time of the left and right heel strikes, and the black vector is the group average vector representing the average of the grey vectors. The heel strikes were clustered around one point of the stimulation cycle (between π/2 and π for the left heel strike). The two plots to the right show the preferred phase and strength of entrainment for each of the separate sequences of 20 s stepping. Here the vectors also point relatively consistently to the same quarter. FIG. 10B shows that no consistent clustering was present in non-responders (P04).

### Faster Alternating DBS Did not Systematically Accelerate Patients' Stepping Rhythm

We also tested if patients' stepping rhythm was faster in the fast alternating DBS condition compared to the slower alternating DBS condition. We performed this comparison across all patients to test if speeding up the stimulation pattern would generally accelerate the stepping rhythm, irrespective of which condition matched their speed more closely. The stepping intervals were not systematically shortened (altDBS=0.55±0.13 s, fast altDBS=0.55±0.14 s, t(9)=−0.3, p=0.806) and the five responders showed changes in either direction (P02: −4.2%, P03: +0.5%; P06: −2.5%, P08: −0.9%; P10: +7.9%).

### Step Timing Variability Remained Similar

First, we compared if the step timing variability changed in the speed-matched alternating DBS condition relative to continuous DBS. No significant differences were found across the ten patients in the coefficient of variation (CV) of the step intervals (contDBS=8.3±3.4%, speed-matched altDBS=9.3±3.2%, t(9)=−0.8, p=0.450) or in the STD of the differences between consecutive step intervals (contDBS=0.07±0.03, speed-matched altDBS=0.07±0.03, t(9)=−0.4, p=0.674).

Next we restricted the analysis to the group of responders, and found that the CV of the step intervals in the speed-matched alternating DBS condition was increased compared to continuous DBS (contDBS=8.2±3.0%, speed-matched altDBS=10.9±3.9%, t(4)=−2.9, p=0.045). This is consistent with a failure of the step cycle to continuously entrain to the alternating stimulation rhythm, leading to increased phase slips as stepping falls in and out of register with the stimulation rhythm. When testing individually in each patient how the step timing variability changed between the stepping sequences recorded in the two continuous and alternating DBS conditions, two of the five patients showed significantly increased variability during alternating DBS (rank-sum test between the respective stepping sequences: P03 p=0.040, P08: p=0.004).

In the group of the five responders, we also compared if their step timing variability differed between the speed-matched and mismatched alternating DBS condition. We found no significant difference across the group (speed-matched altDBS=10.9±3.9%, mismatched altDBS=9.9±2.9%, t(4)=2.1, p=0.101), but in the within-patients tests, one of the responders (P10) had a significantly higher step timing variability when stimulated with mismatched alternating DBS compared to speed-matched alternating DBS (two-samples t-test: t(21)=−2.8, p=0.010).

### Discussion

We found that alternating DBS, that is intermittently lowering and increasing stimulation intensity with an offset between the right and left STN to produce an alternating stimulation pattern, can significantly entrain the stepping rhythm of Parkinson's patients while other motor symptoms remained relatively well controlled. The preferred timing of the steps relative to the stimulation pattern was highly consistent across the patients that significantly entrained to alternating DBS, providing evidence that the STN is mechanistically involved in organising stepping. This is consistent with the alternating pattern of beta activity previously reported in the STN during stepping movements (Fischer et al., 2018), although, by themselves, correlational observations so far could not distinguish between the mechanistic or secondary (afferent) involvement of STN activity (Singh et al., 2013; Fischer et al., 2018; Georgiades et al., 2019).

Our findings also suggest that entrainment only occurs when the stimulation speed closely matches the participants' stepping speed. The faster alternating DBS condition, which was accelerated by 20%, failed to accelerate patients' stepping speed. Amongst responders, alternating DBS could increase patients' step timing variability. Step timing variability would not change if the stepping and stimulation rhythms were aligned only by coincidence. The increase in variability suggests that entrainment was relatively weak and that stimulation can act like an attractor, pulling the intrinsic rhythm in to register, but only intermittently, punctuated by phase slips. The likelihood of phase slips likely depends on how well the alternating stimulation rhythm matches that of natural stepping.

It is acknowledged that stepping in place performance does not necessarily reflect how alternating DBS would affect gait variability during free walking. Despite the instruction to maintain a comfortable stepping movement as consistently as possible, some patients showed considerable variability in how high they lifted their feet across the recording session and even within individual stepping sequences, which in turn affected their step intervals. As we had no recordings of leg kinematics, this could not be quantified or analysed further. We decided to use stepping in place on force plates for the entrainment assessment because it is safer than free walking, could be performed in a relatively small space and provided a simple measure of step timing, which was our main focus in this study. Moreover, the speed of stepping in place appears to match the speed of real walking reasonably well, at least in healthy people (Garcia et al., 2001).

We did not observe any apparent improvement of free walking or reduction of freezing episodes with open-loop alternating DBS applied in this study, but we have now established that the 20 m walking assessment and turning could be safely performed by our cohort. Our study was not optimized for testing potential therapeutic benefits of alternating DBS, but we have now attained a first template for the preferred alignment between alternating DBS and the stepping cycle based on the five responders. This template can be used to inform future studies, in which the stimulation pattern could be aligned to the stepping rhythm as the patient starts walking with the help of external cues or by tracking the stepping rhythm (Tan et al., 2018).

Therefore, in one type of embodiment, the stimulation device 1 may comprise a tracking system arranged to track the gait of the patient. Where a tracking system is used, the stimulation generator 15 of the stimulation device 1 may be configured to generate and apply respective stimulation signals having a rate of electrical energy delivered that is modulated with alternating waveforms at a gait frequency synchronously with the tracked gait.

A stimulation device 1 including an example of a tracking system 20 is shown in FIG. 11 and arranged as follows. The tracking system 1 is configured to receive signals from electrodes 21 implanted in one or more subcortical regions of the brain of the patient, which may be the same or different subcortical regions from those to which the stimulation signals are applied. The tracking system 20 includes a signal processing unit 22 arranged to extract a reference signal such as an LFP (local field potential) signal. The signal processing unit 22 may have a conventional construction. The tracking system 20 also includes a decoding unit 23 arranged to detect features (such as events or phases) of the gait from the reference signal, for example using a Hidden Markov Model or other type of machine learning that may be trained based on reference signals correlated with observed gait. The tracking system 20 may be configured as disclosed in Tan et al., 2018 to which reference is made for further details.

In another example, such a tracking system may include a camera arranged to capture video images of the patient and an image processing unit arranged to detect features of the gait in the video images.

In another example (not shown), such a tracking system may include a worn sensor, such as a wireless motion sensor worn by the patient or pressure sensor installed in insoles of footwear used by the patient, and a processing unit arranged to detect features of the gait in the output of the sensor.

Motion tracking during free walking could also allow examinations of changes in stride length, which could not be assessed in the current study. In real-world situations, the gait cycles adapt to the environment and may vary in timing, hence a more dynamic approach to closed-loop stimulation may be necessary to support free walking. As some freezers experience severe gait initiation problems, it may be helpful to kick-start rhythmic modulation by switching DBS to an alternating mode in an open-loop fashion, but then move into a closed-loop mode as soon as the patient starts walking.

We chose to stimulate at a high intensity for two thirds of the gait cycle and reduce stimulation for one third of the gait cycle, partially because the communication protocol with Nexus-D did not allow a 50-50% stimulation pattern. The preferred alignment for other stimulation patterns or the strength of entrainment may differ.

Two parameters need to be considered carefully when providing alternating DBS: The intensity difference and the overall stimulation intensity. We have shown that even with an intensity difference of only 1V, for example reducing 2.5 and 3.5V to 1.5 and 2.5V, entrainment can occur.

The second parameter, overall stimulation energy, was reduced by one third in our study in the 8 cases for which stimulation intensity was reduced to 0V. We thus delivered considerably less current to the STN than during continuous DBS, which may have lessened our ability to reinforce the stepping cycle and prevent freezing. To match the overall stimulation energy between alternating DBS and continuous DBS, the stimulation boundaries could be shifted upwards to alternate around the clinically effective voltage instead of setting it only lower. However, if the upper threshold is increased, the probability of unwanted side effects would increase too, which would need to be monitored carefully. The side effects observed in the current study were relatively mild and immediately disappeared when stimulation was switched back to the continuous mode.

Alternating stimulation was activated for a limited period of time and it is possible that prolonged stimulation may result in greater deterioration of overall motor symptoms. The clinical benefits of alternating stimulation with respect to gait are likely to be greatest if the alternating stimulation is gait-triggered and gait-limited.

Five of our ten patients did not notice any difference between alternating and continuous DBS. Two of these patients reported that switching DBS off outside of this study did not result in immediately noticeable deterioration of symptoms, and are thus atypical in their response to DBS, but were still included in the analyses. As might be expected, they were not entrained to alternating DBS. For the remaining three patients it is less clear why their stepping was not entrained. The stimulation speed for the non-responders was matched similarly well to their stepping speed as in the group of responders and the severity of gait impairments was similarly variable. The presence of freezing also did not seem to play a role in this comparatively small sample. Also the location of the active DBS contacts did not appear to be critical, considering that in some responders the active contacts were located in the dorsal while in others they were in the ventral part of the STN. The only criterion that stood out was that the patients in the responding group had a stimulation frequency of either 80 or 100 Hz, slightly lower than the conventional stimulation frequency of 130 Hz for STN DBS (Moro et al., 2002). This is interesting considering that several studies suggest that lowering the frequency can be beneficial for improving gait problems in some patients (di Biase and Fasano, 2016; Xie et al., 2018; Di Giulio et al., 2019).

Patients tended to perform the most effortful part of the gait cycle—lifting a foot off the ground—after the contralateral STN had been stimulated at the clinically effective threshold for several hundred milliseconds, which is in line with the known movement-facilitatory effects of DBS. High-intensity stimulation also coincided with the time of the beta rebound, which peaks after the contralateral heel strike according to our previous study. Because STN DBS can counteract excessive beta synchrony (Eusebio and Brown, 2009; Tinkhauser et al., 2017), stimulating with a high intensity after the contralateral heel strike could potentially prevent beta synchronization going overboard in the stance period. Excessive beta synchrony has recently been related to freezing episodes (Storzer et al., 2017; Georgiades et al., 2019) and to the vulnerability to such episodes (Chen et al., 2019), hence stimulating more strongly at points where beta synchronization is more likely may be a more effective stimulation strategy for preventing freezing than continuous DBS.

A recent study also found that non-invasive transcranial alternating current stimulation (tACS) over the cerebellum can entrain the walking rhythm of healthy participants. The STN projects to the cerebellum via the pontine nuclei, thus alternating STN DBS could potentially entrain the gait rhythm via this route (Bostan et al., 2010). The pedunculopontine nucleus (PPN), part of the mesencephalic locomotor region, also is reciprocally connected with the STN, and might provide another pathway by which STN DBS modulates stepping (Jenkinson et al., 2009; Morita et al., 2014; Thevathasan et al., 2018).

Finally, the STN also communicates with the mesencephalic locomotor region through the substantia nigra pars reticulata. The latter structure may be preferentially sensitive to lower stimulation frequencies (Weiss et al., 2019), and it is interesting to note that lower stimulation frequencies tended to be associated with successful entrainment to alternating stimulation in the present study.

In summary, this study provides evidence that the STN is causally important in the dynamic control of the stepping cycle and provides a novel means of modulating this control through alternating STN DBS in patients with Parkinson's disease. This stimulation mode can entrain stepping and parallels the alternating pattern of beta activity recorded in STN during gait. It is expected that this potentially biomimetic stimulation pattern can provide the basis for a novel treatment strategy for patients with debilitating gait disturbances. Our results suggest that it will be key to match the stimulation pattern closely to the patients' preferred walking speed if this is to be reinforced through entrainment.

### REFERENCES

Berens P. CircStat: A MATLAB Toolbox for Circular Statistics. J. Stat. Softw. 2009; 31
di Biase L, Fasano A. Low-frequency deep brain stimulation for Parkinson's disease: Great expectation or false hope? Mov. Disord. 2016; 31: 962-967.
Bostan A C, Dum R P, Strick P L. The basal ganglia communicate with the cerebellum. Proc. Natl. Acad. Sci. 2010; 107: 8452-8456.
Chen C-C, Yeh C-H, Chan H-L, Chang Y-J, Tu P-H, Yeh C-H, et al. Subthalamic nucleus oscillations correlate with vulnerability to freezing of gait in patients with Parkinson's disease. Neurobiol. Dis. 2019; 132: 104605.
Eusebio A, Brown P. Synchronisation in the beta frequency-band—The bad boy of parkinsonism or an innocent bystander? Exp. Neurol. 2009; 217: 1-3.
Fischer P, Chen C, Chang Y, Yeh C, Pogosyan A, Herz D, et al. Alternating modulation of subthalamic nucleus beta oscillations during stepping. J. Neurosci. 2018; 38: 5111-5121.
Garcia R K, Nelson A J, Ling W, Van Olden C. Comparing Stepping-in-Place and Gait Ability in Adults With and Without Hemiplegia. Arch. Phys. Med. Rehabil. 2001; 82: 36-42.
Georgiades M J, Shine J M, Gilat M, McMaster J, Owler B, Mahant N, et al. Hitting the brakes: pathological subthalamic nucleus activity in Parkinson's disease gait freezing. Brain 2019: 3906-3916.
Di Giulio I, Kalliolia E, Georgiev D, Peters A L, Voyce D C, Akram H, et al. Chronic subthalamic nucleus stimulation in Parkinson's disease: Optimal frequency for gait depends on stimulation site and axial symptoms. Front. Neurol. 2019; 10
Jenkinson N, Nandi D, Muthusamy K, Ray N J, Gregory R, Stein J F, et al. Anatomy, physiology, and pathophysiology of the pedunculopontine nucleus. Mov. Disord. 2009; 24: 319-328.
Morita H, Hass C J, Moro E, Sudhyadhom A, Kumar R, Okun M S. Pedunculopontine nucleus stimulation: Where are we now and what needs to be done to move the field forward? Front. Neurol. 2014; 5
Moro E, Esselink R J a, Xie J, Hommel M, Benabid a L, Pollak P. The impact on Parkinson's disease of electrical parameter settings in STN stimulation. Neurology 2002; 59: 706-713.
Singh A, Plate A, Kammermeier S, Mehrkens J H, Ilmberger J, Boetzel K. Freezing of gait-related oscillatory activity in the human subthalamic nucleus. Basal Ganglia 2013; 3: 25-32.
Storzer L, Butz M, Hirschmann J, Abbasi O, Gratkowski M, Saupe D, et al. Bicycling suppresses abnormal beta synchrony in the Parkinsonian basal ganglia. Ann. Neurol. 2017.
Tan H, Fischer P, Shah S A, Vidaurre D, Woolrich M W, Brown P. Decoding Movement States in Stepping Cycles based on Subthalamic LFPs in Parkinsonian Patients. In: 40th Annual International Conference of the IEEE Engineering in Medicine and Biology Society (EMBC). 2018. p. 1384-1387.
Thevathasan W, Debu B, Aziz T, Bloem B R, Blahak C, Butson C, et al. Pedunculopontine nucleus deep brain stimulation in Parkinson's disease: A clinical review. Mov. Disord. 2018; 33: 10-20.
Tinkhauser G, Pogosyan A, Little S, Beudel M, Herz D M, Tan H, et al. The modulatory effect of adaptive deep brain stimulation on beta bursts in Parkinson's disease. Brain 2017; 140: 1053-1067.
Weiss D, Milosevic L, Gharabaghi A. Deep brain stimulation of the substantia nigra for freezing of gait in Parkinson's disease: is it about stimulation frequency? Park. Relat. Disord. 2019; 63: 229-230.
Xie T, Bloom L, Padmanaban M, Bertacchi B, Kang W, MacCracken E, et al. Long-term effect of low frequency stimulation of STN on dysphagia, freezing of gait and other motor symptoms in P D. J. Neurol. Neurosurg. Psychiatry 2018; 89: 989-994.

1. A stimulation device for treatment of gait impairment of a patient, the stimulation device being configured to apply respective stimulation signals to electrodes bilaterally implanted in two subcortical regions of the left and right hemispheres of the brain of the patient, the subcortical regions being associated with motor control,
wherein the stimulation device is configured to apply respective stimulation signals having a rate of electrical energy delivered that is modulated with alternating waveforms at a gait frequency and out of phase with each other.

2. A stimulation device according to claim 1, wherein the stimulation signals have a stimulation frequency of at least 20 Hz, preferably at least 50 Hz.

3. A stimulation device according to claim 1, wherein the stimulation signals have a stimulation frequency of at most 180 Hz.

4. A stimulation device according to claim 1, wherein the stimulation signals have a stimulation frequency that is at least 20% of the frequency of a clinical stimulation signal that is clinically defined for use during continuous chronic stimulation.

5. A stimulation device according to claim 1, wherein the stimulation signals have a stimulation frequency that is at most 140% of the frequency of a clinical stimulation signal that is clinically defined for use during continuous chronic stimulation.

6. A stimulation device according to claim 1, wherein the alternating waveforms have a maximum rate of electrical energy delivered and a minimum rate of electrical energy delivered, the minimum rate of electrical energy delivered being 65% or less of the maximum rate of electrical energy delivered.

7. A stimulation device according to claim 1, wherein the alternating waveforms have a maximum rate of electrical energy delivered that is at least 100% of the rate of electrical energy delivered of a clinical stimulation signal that is clinically defined for use during continuous chronic stimulation.

8. A stimulation device according to claim 1, wherein the alternating waveforms have a maximum rate of electrical energy delivered that is at most 200% of the rate of electrical energy delivered of a clinical stimulation signal that is clinically defined for use during continuous chronic stimulation.

9. A stimulation device according to claim 1, wherein the alternating waveforms have a minimum rate of electrical energy delivered that is at most 65% of the rate of electrical energy delivered of a clinical stimulation signal that is clinically defined for use during continuous chronic stimulation.

10. A stimulation device according to claim 1, wherein the rate of electrical energy delivered of the alternating waveforms remains in the upper quartile of the range between the minimum rate of electrical energy delivered and maximum rate of electrical energy delivered for at least 10% of the period of the alternating waveforms, preferably for at least 20% of the period of the alternating waveforms.

11. A stimulation device according to claim 1, wherein the rate of electrical energy delivered of the alternating waveforms remains in the lower quartile of the range between the minimum rate of electrical energy delivered and maximum rate of electrical energy delivered for at most 90% of the period of the alternating waveforms, preferably for at most 80% of the period of the alternating waveforms.

12. A stimulation device according to claim 1, wherein the rate of electrical energy delivered of the alternating waveforms remains in the upper quartile of the range between the minimum rate of electrical energy delivered and maximum rate of electrical energy delivered for at most 90% of the period of the alternating waveforms, preferably for at most 70% of the period of the alternating waveforms.

13. A stimulation device according to claim 1, wherein the rate of electrical energy delivered of the alternating waveforms remains in the lower quartile of the range between the minimum rate of electrical energy delivered and maximum rate of electrical energy delivered for at least 10% of the period of the alternating waveforms, preferably for at least 30% of the period of the alternating waveforms.

14. A stimulation device according to claim 1, wherein the stimulation device is configured to apply respective stimulation signals having the rate of electrical energy delivered modulated by modulation of an amplitude of the stimulation signals.

15. A stimulation device according to claim 1, wherein the stimulation device is configured to apply respective stimulation signals having the rate of electrical energy delivered modulated by modulation of a stimulation frequency of the stimulation signals.

16. A stimulation device according to claim 1, wherein the stimulation device is configured to apply respective stimulation signals having the rate of electrical energy delivered that is modulated by modulation of a pulse width of the stimulation signals.

17. A stimulation device according to claim 1, wherein the stimulation device is configured to apply respective stimulation signals having the rate of electrical energy delivered modulated by modulation of at least two of: an amplitude of the stimulation signals; a stimulation frequency of the stimulation signals; and a pulse width of the stimulation signals.

18. A stimulation device according to claim 1, wherein the alternating waveforms are out of phase by 50% of the period of the alternating waveforms.

19. A stimulation device according to claim 1, wherein the alternating waveforms are square waveforms.

20. A stimulation device according to claim 1, wherein the stimulation signals have identical alternating waveforms.

21. A stimulation device according to claim 1, wherein the gait frequency is a desired gait frequency.

22. A stimulation device according to claim 21, wherein the desired gait frequency is a gait frequency measured in the absence of stimulation or is selected in the physiological range for an age group of a patient.

23. (canceled)

24. A stimulation device according to claim 1, wherein the stimulation device further comprises a tracking system arranged to track the gait of the patient and the stimulation device is arranged to apply stimulation signals having a rate of electrical energy delivered that is modulated with alternating waveforms at a gait frequency synchronously with the tracked gait.

25. A stimulation device according to claim 1, wherein the subcortical regions are the subthalamic nuclei or the pedunculopontine nuclei.

26. (canceled)

27. A stimulation device according to claim 1, wherein the patient is a patient with Parkinson's Disease, Progressive Supranuclear Palsy or Multiple System Atrophy.

28. A stimulation device according to claim 1, further comprising electrodes for bilateral implantation in the two subcortical regions of the left and right hemispheres of the brain of the patient.

29. A method of treatment of gait impairment of a patient, the method comprising applying respective stimulation signals to electrodes bilaterally implanted in two subcortical regions of the left and right hemispheres of the brain of the patient, being regions associated with motor control, the respective stimulation signals being amplitude modulated by alternating waveforms at a gait frequency and out of phase with each other.
